(19) United States
(12) Patent Application Publication
(10) Pub. No.: US 2007/0023042 A1
(43) Pub. Date: Feb. 1, 2007

(54) Demand free air flow inhalator

(76) Inventor: Lanny R. Lee, Toms River, NJ (US)

Correspondence Address: LANNY R. LEE, STE. 324, 1358 HOOPER, TOMS RIVER, NJ, 08753-2882, US

(21) Appl. No.: 11/136,784

(22) Filed: Jul. 28, 2005

(51) Int. Cl. A61M 15/00; B65D 83/06

(52) U.S. Cl. 128/203.15

(57) ABSTRACT

An inhalation device for administering powered forms of medicament includes a mouthpiece connected to a first closable port suction chamber with at least one deformable wall separating a second chamber. First closable port having a throat is sealed with a cover and a structure for locating at least one medicament powder pouch and a means of exposing contents. A carousel to include a plurality of powder pouches arrayed radial and a device to index each before exposing contents before an exhaust port communicating before a second chamber is also described.

### FIELD AND THE BACKGROUND OF THE INVENTION

Inhalators have been part of medicine used to spray liquid and powered medicament orally for treating nose, throat and lung problems. Sprayed medicament that are sprayed into an inhaled air stream as seen in U.S. Pat. No. 6,866,037 and U.S. Pat. No. 6,712,070 have a much higher velocity than the inhaled air resulting in much of the medicament coats the back of the throat. It is absorbed into the system which is often undesirable. The powered medicament is aerated by turbulence created by inhaling the air through restricted passages, then carried to the lungs in the inhaled air stream. This back pressure slows the air intake and allows much of these, less than 20 Mcg dose, of micron size particles to coat the throat before the patient is able to accelerate the air stream. Another difficulty encountered is when the user must expand their lungs rapidly from rest while avoiding blowing into the inhalation device as this will cause a loss of medicament into the outside air. All of these problems and limitations of these present devices are exacerbated because the user has a respiratory disorder. As the users are often have poor eyesight along with having poor motor skills handling the small capsules is a very real problem of present devices. Other devices are not refillable and are a major cost driver to already expensive medicaments; others that contain fluorocarbon propellants will soon be banned from sale. A major disadvantage of prior art is: there is no means provided for the user to confirm that the inhalation device has properly dispensed the dosage as in U.S. Pat. No. 6,792,945.

### BRIEF SUMMARY OF THE INVENTION

An inhalation device for administering powered forms of medicament includes a mouthpiece connected to a first closable port suction chamber with at least one deformable wall separating a second chamber having at least one port to allow outside air entry and at least one exhaust port to the first chamber. Closable port having a throat is sealed with a cover and a means for locating at least one medicament powder pouch and a means of exposing contents. A carousel is also described to include a plurality of medicament powder pouches arrayed radial and a mechamism to index each before exposing means. The cover is hinged to seal first chamber inlet port and also the exhaust of the second chamber is sprung to resist against a latch having a drawbar connected centrally to deformable wall. When adequate suction is applied by the user to first chamber and deformable wall has excursion into the first chamber by sufficient pressure differential, drawbar is urged to trip latch. Whereby, cover is sprung open covering intake port and exposing exhaust port provided in the second chamber. Thus second chamber becomes an air pump exhausting air into opened pouch mixing medicament powder into the stream of air rushing in to equalize the pressure of the first chamber. The user can inhale against the resistance of the mechanism and have inertia of the inhaling while feeling increased resistance just before the cover trips. The rush of air to the user is desirable psychologically and physically. The user can view the operational status of the inhalator by looking in the viewing window and see the indicator dial advance. As a further verification the carousel can easily be inspected visually. Although it is unlikely that medicament particles would remain after each cycle, the pouches continue to be exposed to the inhaled stream of air rushing by. A second inhalation device is operated by a pressure differential that trips a cover to open device. In a similar manual device a button is used to unlatch cover.

### BRIEF DESCRIPTION OF THE DRAWINGS

FIG. 1 is a sectional side view of a first inhalation device in a free state in accord with the present invention;

FIG. 2a is a sectional side view of a first inhalation device in FIG. 1 a free state;

FIG. 2b is a sectional side view of a first inhalation device in FIG. 1 in an extended state;

FIG. 3 is an end view of a first inhalation device;

FIG. 4 is an enlarged sectional side view of a first inhalation device in a free state in accord with the present invention;

FIG. 5 is an enlarged partial prospective view of a first inhalation device;

FIG. 6 is an end view of a first inhalation device;

FIG. 7 is an enlarged partial sectional side view of a first inhalation device in an extended state;

FIG. 8 is an isometric partial view of a first inhalation device;

FIG. 9 is a prospective view;

FIG. 10 is an enlarged partial view of FIG. 9;

FIG. 11 is partial view of FIG. 10 rotated slightly counter clockwise;

FIG. 12 is a side view of a first inhalation device;

FIG. 13 is a section end view of a first inhalation device;

FIG. 14 is an isometric view of a medicament pouch carousel assembly;

FIG. 15 is a section view of a inhalation device;

FIG. 16 is an enlarged partial prospective view;

FIG. 17 is an isometric view a second inhalation device;

FIG. 18 is a mouthpiece end view of a second inhalation device;

FIG. 19 is an intake end view of a second inhalation device;

FIG. 20 is an oblique section view of a second inhalation device;

FIG. 20a is an enlarged view;

FIG. 21a is a section view in closed state;

FIG. 21b is a section view in transition to open state;

FIG. 21c is a section view in open state;

FIG. 22 is a plan view of a second inhalation device;

FIG. 23 is an enlarged partial view transition to open;

FIG. 24 is an enlarged partial view closed;

FIG. 25 is an enlarged partial button release;

### DETAILED DESCRIPTION OF THE INVENTION

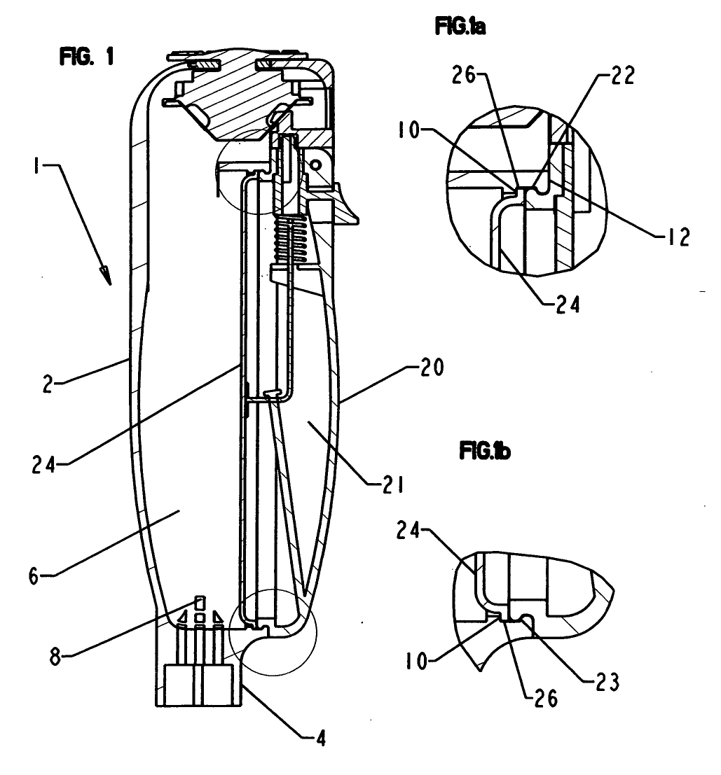

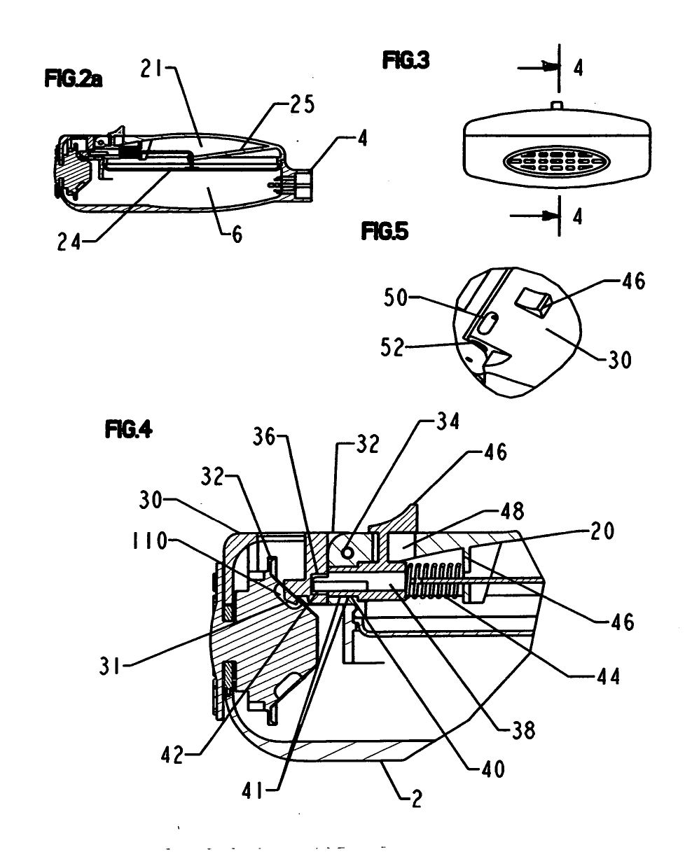

FIGS. 1, 2a and 2b show a first inhalation device I herein comprising an inhalator a first chamber housing 2 having a mouthpiece 4 commuting to first chamber 6 through strainer 8 and an annular rim 12 having a concentric seat face 10. A second chamber housing 20 having a second chamber 21 is attached to the first chamber housing by snap fit mechanism 22, 23 providing a clamp fit and mostly sealed to elastic membrane 24 along flange 26. FIG. 2a shows a side section view of a first inhalation device I showing elastic membrane 24 in the relaxed state and enlarged section is seen in FIG. 4 cover 30 in the second position with hinge 32 and connected to second chamber housing 20 through pivot pin 34 and latch mortise 36 restricting air flow through exhaust port 38 of latch draw bar 40 slidably located in slot 41 in a second chamber housing 20 with latch tongue 42 biased into latch mortise 36 by a compression spring 44 having rest 46. Manual release 46 is given lineal clearance by slot 48 for a lineal freedom. FIG. 5 is a partial view showing intake port 50 and torsion spring 52 under load by cover 30 in closed position. A plow 31 is seen protruding into undercut 110 of medication carousel 100. A transfer bar 25 pivots a inside of housing.

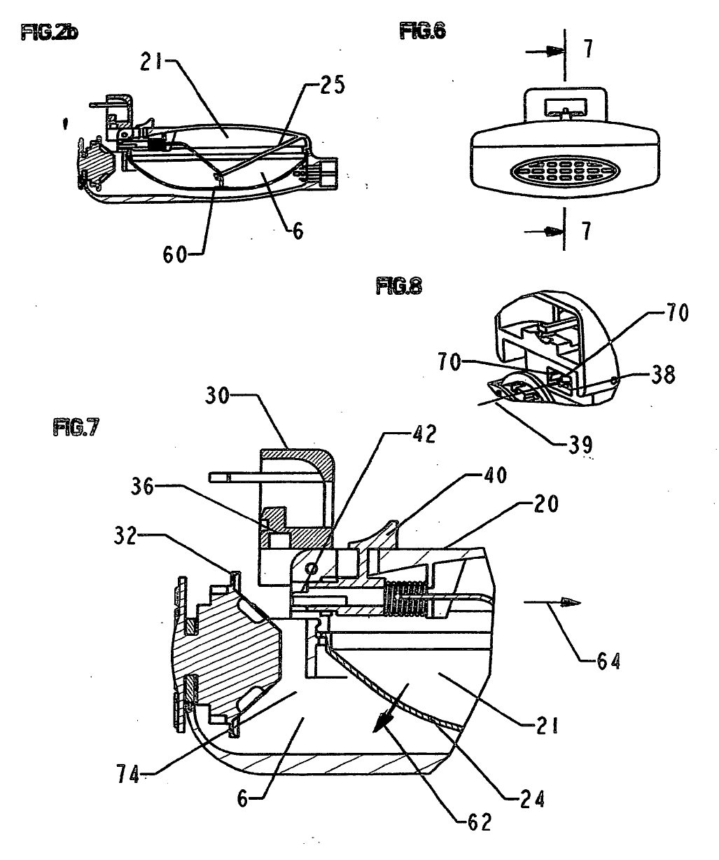

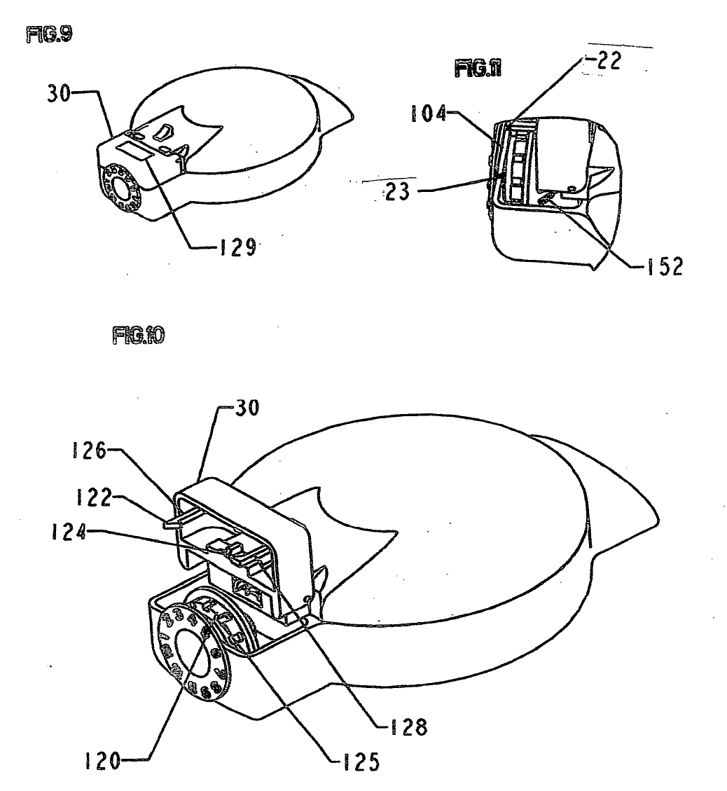

FIG. 2b is a section side view of a first inhalation device shown with a pressure differential resulting from a pressure resistively lowered though mouth piece 4 causing a negative pressure in a first chamber 6 while being partially equalized in second chamber 21 by entry of free air into intake port 50 pushing elastic membrane 24 in first chamber 6. FIG. 7 shows latch draw bar 40 ending with end pad 60 by means connected centrally to elastic membrane 24 is drawn with it a in direction of vector arrow 62 and being translated to urge latch draw bar 40 in direction shown by arrow 64 whereby latch tongue 42 is withdrawn from latch mortise 36 freeing cover 30 to a first position driven by torsion spring 52 thereby exposing exhaust port 38 to permit air flow from second chamber 21. A curved vane 70 causes a vortex stream 74 of air being expelled from a second chamber 21 further being diverted by baffle ring 32 while a main body of inhaled air rushes in and draws vortex air stream 74 along. FIG. 8 is an enlarged view showing exhaust port 38 and internal vane 70 to induce a turbulent air flow 39

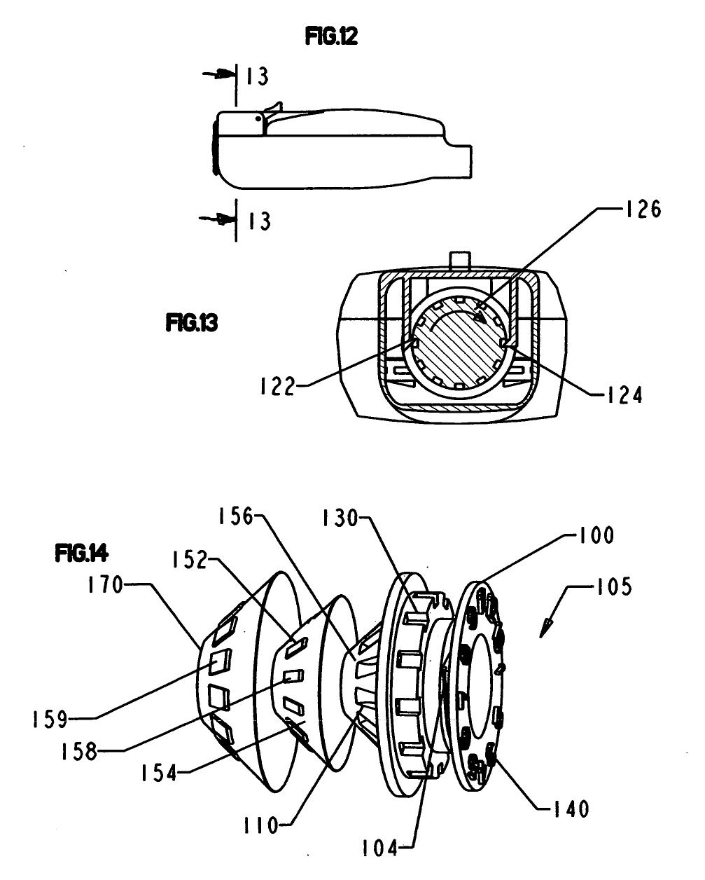

FIG. 9 is a first inhalation device 1 shown in full isometric view in the state of the cover 30 in second position showing view window 129 while FIG. 10 is the same as FIG. 11 in the condition with cover 30 pivoting to a first position. An escapement mechanism 120 comprising of gear 125 along with first drive paw 122, second drive paw 124 which both are connected to cover 30 by first flexible shaft 126 and flexible shaft 128 respectively. FIG. 11 is a partial view where medication pouch 152 is visible along with medication carousel 100 in place with snap mechanism 106, 108 retains insert 104.

FIG. 12 is a first inhalation device 1 shown in full side view and a side view FIG. 13 is a section with first paw 122 on gear tooth 130 to rotate medication carousel 100 one half pitch in direction shown 126 when cover 30 unlatched. When cover 30 is by means closed to latch medication carousel 100 is rotated one half pitch by second paw 124 thereby providing escapement mechanism 120.

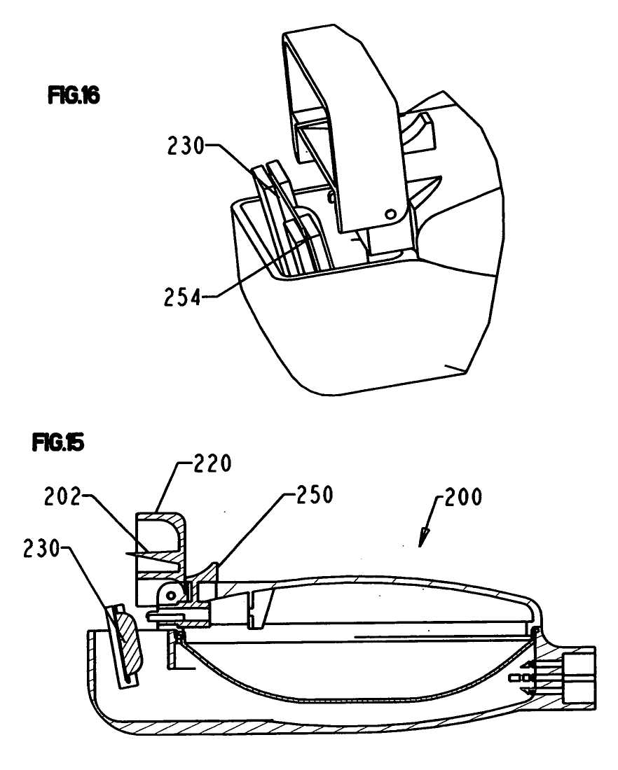

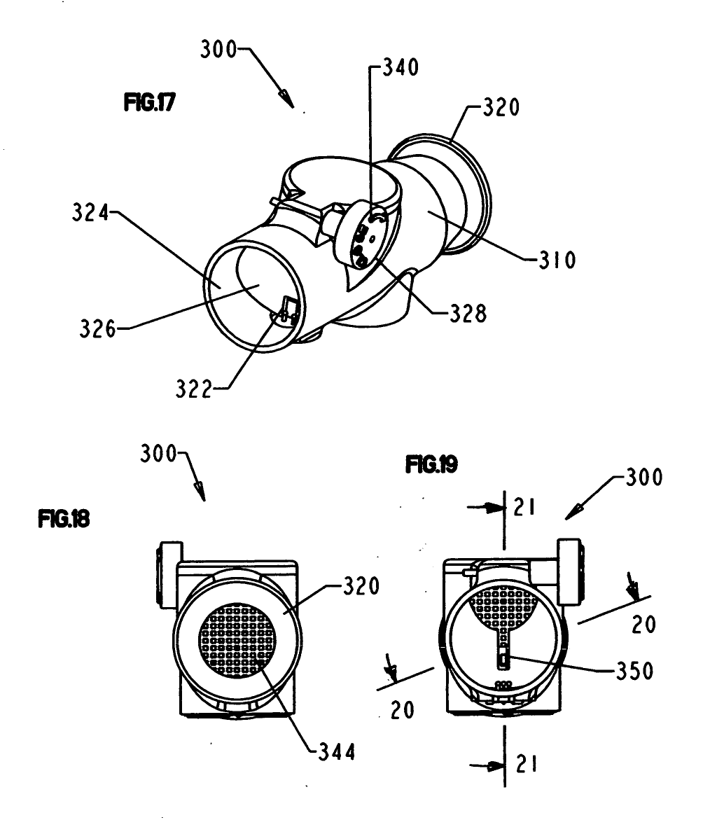

FIG. 14 is a medicament carousel assembly 105 in isometric view showing indicator notes 140 on medication carousel 100 along with medicament carrier 154 having radial spaced medicament containers 152 having rippable covering 158 and preferably removably located to surface 156 and medicament container 152 aligned to undercut 110 A transit protective shield 170 having radial blisters 159 is aligned to medicament containers 152.

FIG. 15 is a second inhalation device 200 shown in section while in suction state and plow 202 poised as shown. When cover 220 is by means closed plow 202 will rip open single pouch 230. Second inhalation device 200 is manually released by 250. Single pouch 230 is removably located by sliding in groove 254.

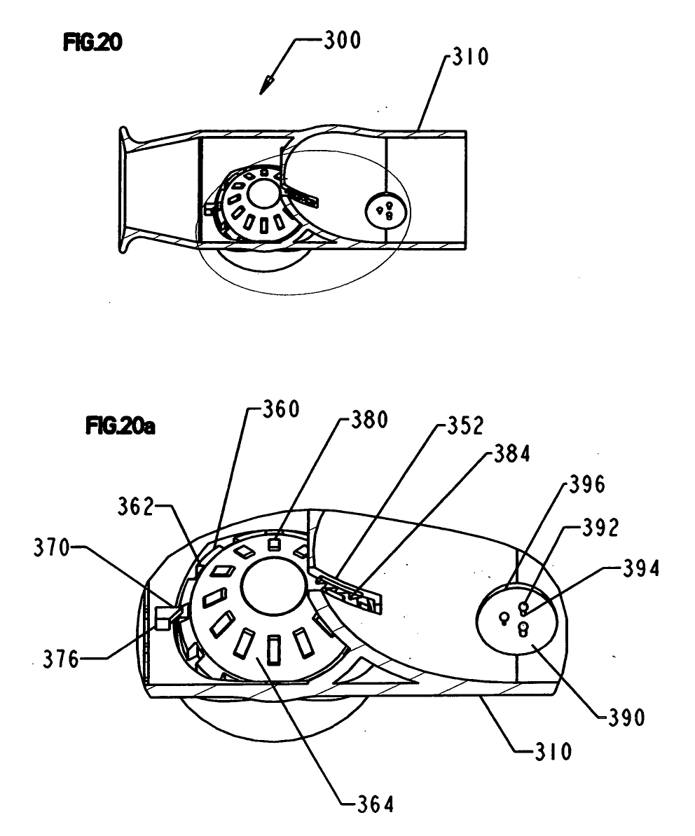

FIG. 17 is an isometric view of a second inhalation device 300 with body 310 including a mouth piece opening 320 and a inlet 324 closed by cover 326 having air duct 322 whereby operated by means to knob 328 showing instructions 340. FIG. 18 an end view showing partition screen 344 before mouthpiece 320. FIG. 19 is inlet end shown in an open state viewing air slot 350.

FIG. 20 is an oblique section view and enlarged view shows a medicament carousel 360 having radial gear tooth 362 and medicament carrier 364 with one or more medicament container packets 380. Ratchet paw 370 is connected to flexure 376 providing stepped unidirectional turning. At least one tine 384 is arrayed along air slot wall 352. Detent ball 392 in connected by flexure 394 to dial plate 396 providing a breakaway selector device 390 having one or more flexure 384 each with incremental stiffness values.

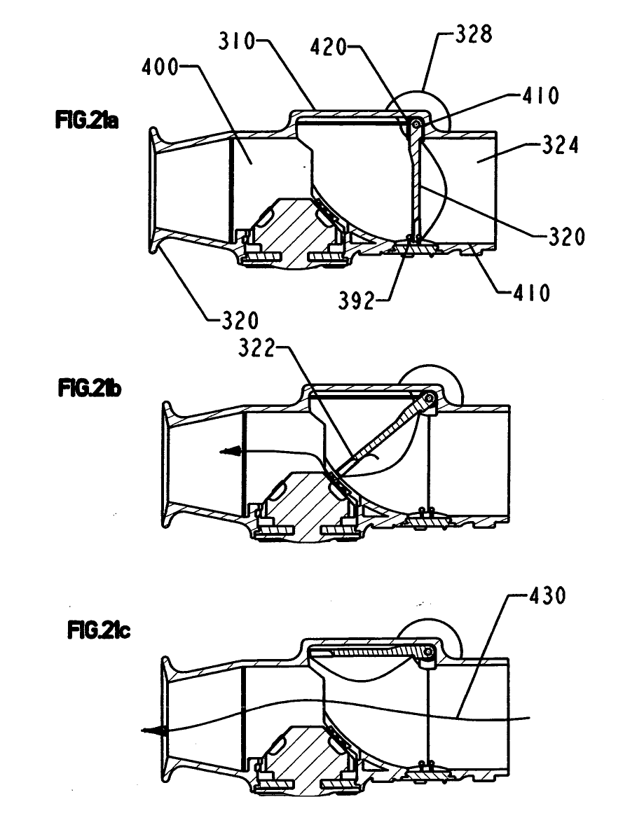

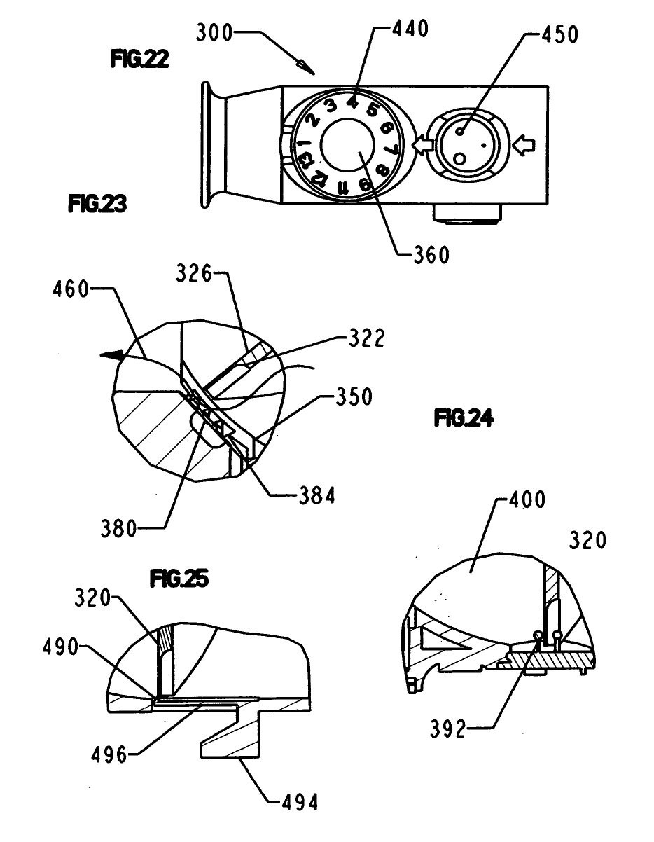

FIG. 21a demonstrates a section of a closed inhalator showing cover 320 closely fit to the inside surface 410 of body 310 pivotally connected by pin 410 to body 310 and is closed using knob 328. Said cover 320 is biased to rest along detent ball 392 by torsion spring 420. Pressure is by means reduced through mouthpiece 320 resulting in a negative pressure in closed chamber 400. When sufficient pressure differential with the assistance of torsion spring 420 to cause cover 320 to overcome detent 392 and pivot on pin 410 and pivot to a first position. FIG. 21b shows cover 320 in transition to a first position and FIG. 21c shows cover 320 at a first position permitting free flow of air 430. FIG. 22 is a plan view showing a second inhalation device 300 and medicament carousel 360 having dose indices 440 and breakaway indices 450. FIG. 23 is an enlarged partial view showing air flow 460 only through air duct 322 to air slot 350 thereby aerating medicament container 380. FIG. 24 is an enlarged view of cover 320 in a second position maintained by detent ball 392. FIG. 25 is an enlarged view of manual lever 496 having release bar 494 retaining cover 320 by dog 490.

What is claimed is:

1. An inhalation device for use with a medicament pack to be received in the device, in which pack a plurality of containers for medicament in powder form are defined, wherein the containers are spaced radial around a axis of and defined between two sheets secured to each other, the device comprising
A body with a axis of rotation with a axis of symmetry aligned and at approximate right angle definition which is generally disc shape having a side with an first opening commuting to a first chamber and an intake port symmetrically opposed having a pivotally connected cover with a latch mechanism and an elastic wall separating second chamber having at least one inlet port and at least one exhaust port;
An indexable medicament carousel with indexing device is disposed in a compartment between first chamber and pivotal cover that has a feature that covers the exhaust port of second chamber when in a first position and covers the intake port when in a second position;
A latch actuation mechanism by means disposed in second chamber connected to elastic wall whereby when by means air pressure lowered in first chamber thereby causing a pressure differential drawing elastic wall into first chamber urging with it connected mechanism thereby tripping a latch whereby cover pivots by means biased to first position;
A protrusion slits rips open a medicament container while turbulence induced air flows from a exhaust port of second chamber directed into container thereby medicament powder is aerated and mixes with air stream moving to the low pressure of the first chamber then on through first opening to the low pressure source;
A screen means is disposed before first opening to prevent objects from exiting first opening.

2. A device according to claim 1, further compromising a dose monitor indicia indicating a plurality of medicament containers.

3. A device according to claim 1, wherein at least one of the two sheets is of a see through material.

4. A device according to claim 1, resistance to effort required to trip latch is varied by changes in elastic wall thickness.

5. A device according to claim 1, whereby pivotal cover is of a see through material.

6. A device according to claim 1, whereby pivotal cover has a viewing window.

7. A device according to claim 1, whereby medicament carousel is removable to renew medicament pack.

8. A device according to claim 1, whereby medicament pack is manual loaded singular dose container.

9. An inhalation device for use with a medicament pack to be received in the device, in which pack a plurality of containers for medicament in powder form are defined, wherein the containers are spaced radial around a axis of and defined between two sheets secured to each other, the device comprising
A body generally tube shape with a first opening commuting to an intake opening and having a spur passage having a air slot with one or more tines along slot facing first opening and ending within a external bulge;
A intake opening closed by pivotal connected cover having a narrow air duct facing intake opening is biased by means to follow a spur passage and rest in a second position in bulge and a first position maintained by detent device thereby a first chamber is created;
An indexable medicament carousel with indexing device is disposed in first chamber and when by means rotated is having tine rip open medicament container exposing medicament powder;
A negative pressure is my means applied to first opening whereby air pressure is lowered in first chamber thereby causing a pressure differential when it is sufficient the cover overcomes detent mechanism and moves to a second position position;
And part way through arc a cover air duct and air slot align momentarily to allow a rush of air to enter medicament container thereby aerating the contents and as cover continues to second position a body becomes fully open to air stream moving through first opening to low pressure source;
A screen means is disposed before the first opening to prevent objects from exiting first opening.

10. A device according to claim 9, further compromising a dose monitor indicia indicating a plurality of medicament containers.

11. A device according to claim 9, wherein at least one of the two sheets is of a see through material.

12. A device according to claim 9, resistance to effort required to trip latch is varied by changes in detent flexure stiffness.

13. A device according to claim 9, body is of a see through material.

14. A device according to claim 9, body has a viewing window.

15. A device according to claim 9, whereby medicament carousel is removable to renew medicament pack.

16. A device according to claim 9, whereby medicament pack is manual loaded singular dose container.
